(19) United States
(12) Patent Application Publication
(10) Pub. No.: US 2021/0251983 A1
(43) Pub. Date: Aug. 19, 2021

(54) METHODS OF TREATMENT WITH BUPRENORPHINE

(71) Applicant: BioDelivery Sciences International, Inc., Raleigh, NC (US)

(72) Inventors: Thomas Smith, Edisto Island, SC (US); Mancia Ko, Lakewood, CO (US); Todd Kunkel, Overland Park, KS (US)

(73) Assignee: BioDelivery Sciences International, Inc., Raleigh, NC (US)

(21) Appl. No.: 17/175,172

(22) Filed: Feb. 12, 2021

Related U.S. Application Data
(60) Provisional application No. 62/976,222, filed on Feb. 13, 2020.

(51) Int. Cl. A61K 31/485; A61K 9/00; A61P 29/00

(52) U.S. Cl. CPC A61K 31/485; A61P 29/00; A61K 9/006

(57) ABSTRACT

The present disclosure relates to methods of treatment with buprenorphine.

### CROSS-REFERENCE TO RELATED APPLICATIONS

This application claims priority to U.S. Provisional Application No. 62/976,222, filed Feb. 13, 2020. The entire contents of this application are incorporated herein by reference.

### FIELD OF THE TECHNOLOGY

The present disclosure relates to a method of treatment with buprenorphine, including administering an effective amount of buprenorphine to a subject.

### BACKGROUND

Chronic pain is pain that persists beyond the expected healing time, if resulting from injury, and can progress from a bothersome nuisance to a profound affliction. Chronic pain can also be caused by diseases such as osteoarthritis; chronic lower back pain; chronic pelvic pain (CPP); neuropathic pain; fibromyalgia; postoperative pain; central nervous system pain-related pain disorder; Reflex Sympathetic Dystrophy Syndrome (RSDS); repetitive stress injuries; shingles; headaches; or pain related to burns, cancer or lung disease. Chronic pain can cause a marked alteration in behavior with depression and anxiety, restriction in daily activities and excessive use of medication and medical services in an afflicted individual. The treatment of chronic pain is difficult, often inadequate, and associated with high economic and psychological cost.

Opioids are commonly used for long term treatment of chronic pain. The opioid crisis and danger associated with the use of opioids have led to increased scrutiny of prescriptions for chronic pain management. There is potential for abuse with opioids, which can lead to addiction, accidental overdose, and potentially death. A significant portion of opioid-related deaths are caused by respiratory depression.

Buprenorphine is a Schedule III atypical opioid. Buprenorphine is a partial μ-opiate receptor agonist, an ORL1/nociceptin receptor agonist, and a κ-opiate receptor antagonist. Buprenorphine is metabolized by the liver, via both glucuronidation and CYP3A4-mediated N-dealkylation, into norbuprenorphine and other metabolites. Buprenorphine has a low oral bioavailability due to very high first-pass metabolism.

Buprenorphine is available commercially as Temgesic® 0.2 mg and 0.4 mg sublingual tablets, and as Buprenex® in a 0.3 mg/ml parenteral formulation. Buprenorphine is also available as a sublingual preparation (Subutex®) and as a sublingual abuse-resistant formulation with naloxone (Suboxone®). The FDA approved Suboxone/Subutex in 2002 as a treatment of opioid dependence. Sublingual buprenorphine has been used for opioid detoxification and maintenance.

An open-label study used sublingual buprenorphine/naloxone (Suboxone®) for the treatment of chronic pain for chronic opioid users (Malinoff et al., 2005, American Journal of Therapeutics 12, 379-384). Patients were treated with daily buprenorphine doses that ranged from 2-20 mg (mean 8 mg). The treatment lasted from 2.4 months to 16.6 months (mean 8.8 months). The article reports that patients using sublingual buprenorphine experienced improvement in their condition and reported a decrease in their sensation of pain.

### SUMMARY

When the pain cannot be adequately controlled with treatment by non-steroid anti-inflammatory drugs, opioid treatment is typically used for pain management. Standard mu agonist opioids, such as hydrocodone or fentanyl, are often used as a first line therapy. These standard mu agonist opioids present potential for abuse and respiratory depression. Buprenorphine is not currently a first line therapy. However, buprenorphine shows minimal respiratory depression, is effective for treating pain, and is also less addictive than other first line therapies, such as standard mu agonist opioids. The present disclosure describes how buprenorphine should be considered as a first line therapy instead of considering transitioning to buprenorphine if patients on high opioid dosages are unable to taper despite worsening pain and/or function with opioids, whether or not opioid use disorder criteria are met.

In an embodiment, a method for the management of pain severe enough to require daily long term opioid treatment comprises: administering at least 75 μg of buprenorphine transmucosally once daily, as a first line opioid therapy, wherein the pain is managed without substantial respiratory depression.

In an embodiment, administering buprenorphine comprises administering a buprenorphine buccal film. The buprenorphine buccal film comprises buprenorphine or buprenorphine and naloxone.

In an embodiment, from about 300 μg of buprenorphine to about 900 μg of buprenorphine is administered transmucosally once daily. In an embodiment, greater than 900 μg of buprenorphine is administered transmucosally once daily. The buprenorphine can be administered once daily or twice daily.

In an embodiment, the respiratory depression is evaluated by measuring the ventilatory response to hypercapnia through assessment of a decrease in maximum minute ventilation (Eₘₐₓ), relative to placebo, after administration of each dose of buprenorphine. Respiratory depression is indicated when a mean difference in Eₘₐₓ, relative to placebo is clinically insignificant.

In an embodiment, the pain that is managed is pain associated with one or more of the flowing diseases: chronic pelvic pain (CPP); diabetic peripheral neuropathy (DPN); fibromyalgia; postoperative pain; central nervous system pain-related pain disorder; or pain related to burns, cancer or lung disease.

In an embodiment, a method for treating a patient with an opioid while reducing the risk of opioid addiction comprises: administering an effective amount of buprenorphine as a first line opioid therapy to treat a disease, a symptom of a disease, or a condition of a patient; and reducing the risk of opioid addiction by maintaining an abuse quotient of buprenorphine below 1, wherein the abuse quotient is defined as Cₘₐₓ/Tₘₐₓ.

In an embodiment, administering buprenorphine comprises administering a buprenorphine buccal film. The buprenorphine buccal film comprises buprenorphine or buprenorphine and naloxone.

In an embodiment, at least 75 μg of buprenorphine transmucosally once daily is administered transmucosally once daily to treat a disease, a symptom of a disease, or a condition of a patient. In an embodiment, from about 300 μg of buprenorphine to about 900 μg of buprenorphine is administered transmucosally once daily to treat a disease, a symptom of a disease, or a condition of a patient. In an embodiment, greater than 900 μg of buprenorphine is administered transmucosally once daily to treat a disease, a symptom of a disease, or a condition of a patient. The buprenorphine can be administered once daily or twice daily.

In an embodiment, the disease, the symptom of the disease, or the condition of the patient treated comprises at least one of osteoarthritic pain; chronic lower back pain; chronic pelvic pain (CPP); neuropathic pain; fibromyalgia; postoperative pain; central nervous system pain-related pain disorder; Reflex Sympathetic Dystrophy Syndrome (RSDS); repetitive stress injuries; shingles; headaches; or pain related to burns, cancer or lung disease.

In an embodiment, a method of treating post-traumatic stress disorder (PTSD) or a symptom of PTSD comprises: administering an effective amount of buprenorphine to a subject having PTSD, wherein PTSD or a symptom of PTSD is treated. In an embodiment, administering the effective amount of buprenorphine comprises transmucosally administering the effective amount of buprenorphine.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a graph of potential for respiratory depression vs. dose for full μ-opioid agonists and buprenorphine.

FIG. 2 depicts a schematic diagram of the testing parameters for groups in a study for respiratory depression of opioids.

FIG. 3 depicts a flow chart of study procedures before and during testing.

FIG. 4 depicts minute ventilation as a function of end-tidal CO₂ two hours post-dose, for a single study subject.

FIG. 5 depicts a bar graph summary of the LS mean difference for each treatment.

FIG. 6 depicts the mean minute ventilation over time for each study drug.

FIG. 7 depicts relative minute ventilation (with respect to placebo) vs. time for all study completers.

FIG. 8 depicts oxygen saturation vs. time for each study drug.

FIG. 9 depicts data collected for the relative Peak Expiratory Flow Rate (PEFR), with respect to placebo vs. time for all study completers.

FIG. 10 depicts a graph of mean pupil diameter vs. hours (post-dose) for BBF 300 μg, BBF 600 μg, BBF 900 μg, oxycodone 30 mg, and oxycodone 60 mg.

FIG. 11 depicts pupil diameter (mm) LS mean change from baseline over time for BBF 300 μg, oxycodone 30 mg, and oxycodone 60 mg.

FIG. 12 depicts pupil diameter (mm) LS mean change from baseline over time for BBF 600 μg, oxycodone 30 mg, and oxycodone 60 mg.

FIG. 13 depicts pupil diameter (mm) LS mean change from baseline over time for BBF 900 μg, oxycodone 30 mg, and oxycodone 60 mg.

FIG. 14 depicts graphs of mean plasma concentration vs. time for BBF and IR Oxycodone.

FIG. 15 shows the % of patients that experienced treatment-emergent adverse events.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION

The following definitions are provided as guidance as to the meaning of certain terms used herein.

As used herein, the articles “a” and “an” mean “one or more” or “at least one,” unless otherwise indicated. That is, reference to any element of the present disclosure by the indefinite article “a” or “an” does not exclude the possibility that more than one of the element is present.

As used herein, the term “acute pain” refers to pain characterized by a short duration, e.g., three to six months. Acute pain is typically associated with tissue damage, and manifests in ways that can be easily described and observed. It can, for example, cause sweating or increased heart rate. Acute pain can also increase over time, and/or occur intermittently.

As used herein, the term “bioavailability” is as defined in 21 CFR Section 320.1 and refers to the rate and extent to which the active ingredient or active moiety is absorbed from a drug product and becomes available at the site of action. The term “bioavailability”, “absolute bioavailability” or “total bioavailability” refers to the total bioavailability including amounts that are absorbed through the oral mucosal membrane (i.e., transmucosally) and through the GI mucosa of the lower GI tract. In some embodiments, the transmucosal drug delivery devices of the present disclosure provide bioavailability of buprenorphine of between 65% and 85%. In some embodiments, the bioavailability of buprenorphine is 80%.

As used herein, the term “chronic pain” refers to pain which persists beyond the usual recovery period for an injury or illness. In some embodiments, chronic pain is a pain that lasts longer than one week. Chronic pain can be constant or intermittent. Common causes of chronic pain include, but are not limited to, arthritis; chronic lower back pain; chronic pelvic pain (CPP), diabetic peripheral neuropathy (DPN), fibromyalgia, postoperative pain, central nervous system pain-related pain disorder, Reflex Sympathetic Dystrophy Syndrome (RSDS); repetitive stress injuries; shingles; headaches; or pain related to burns, cancer or lung disease.

As used herein, the term “chronic low back pain” refers to a musculoskeletal disorder, wherein the subject experiences pain in the lumbar, or low back region for at least 12 weeks. In some embodiments, a subject experiences chronic low back pain for at least 3 months.

As used herein, the term “neuropathic pain” refers to a complex, chronic pain that usually is accompanied by tissue injury and results from lesions or diseases affecting the somatosensory system. With neuropathic pain, the nerve fibers themselves may be damaged, dysfunctional or injured. These damaged nerve fibers send incorrect signals to other pain centers. The impact of nerve fiber injury includes a change in nerve function both at the site of injury and areas around the injury. An example of neuropathic pain is diabetic peripheral neuropathy (DPN).

As used herein, the term “osteoarthritic pain” refers to pain resulting from osteoarthritis, a degenerative joint disease and the most common type of arthritis. It is associated with the degradation and loss of a cartilage that covers and cushions the ends of bones in normal joints. Osteoarthritis causes the cartilage in a joint to become stiff and lose its elasticity, making it more susceptible to damage. Over time, the cartilage may wear away in some areas, greatly decreasing its ability to act as a shock absorber. As the cartilage wears away, tendons and ligaments stretch, causing pain. If the condition worsens, the bones could rub against each other, causing even more pain and loss of movement.

As used herein, unless indicated otherwise, the term “buprenorphine”, includes any pharmaceutically acceptable form of buprenorphine, including, but not limited to, salts, esters, and prodrugs thereof. As used herein, the term “buprenorphine derivative” refers to compounds having similar structure and function to buprenorphine. In some embodiments, buprenorphine derivatives include those of the following formula:

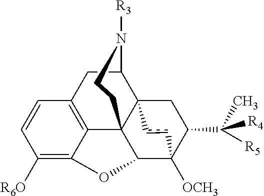

or pharmaceutically acceptable salts or esters thereof, wherein

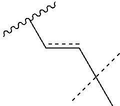

is a double or single bond; R₃ is selected from a —C₁₋₄ alkyl group or a cycloalkyl-substituted-C₁₋₄ alkyl group; R₄ is selected from a —C₁₋₄ alkyl; R₅ is —OH, or taken together, R₄ and R₅ form a═O group; and R₆ is selected from —H or a —C₁₋₄ alkyl group.

Buprenorphine derivatives include, but are not limited to, etorphine and diprenorphine. Formulations of buprenorphine and buprenorphine derivatives are described in International Application Publication No. WO 2008/011194, which is hereby incorporated by reference.

As used herein, unless indicated otherwise, the term “naloxone” includes any pharmaceutically acceptable form of naloxone, including, but not limited to, salts, esters, and prodrugs thereof.

As used herein, “non-parenteral” refers to modes of administration other than by direct systemic delivery of the medicament. As such, “non-parenteral” excludes the use of intravenous (IV) injection, intramuscular (IM) injection, Intraperitoneal (IP) injection, subcutaneous (SC) injection, etc. for administration of the medicament and includes transdermal, oral transmucosal administration, and administration via the GI tract, generally.

As used herein, the term “mucoadhesive layer” or “polymeric diffusion environment” refers to an environment capable of allowing flux of a medicament to a mucosal surface upon creation of a gradient by adhesion to a mucosal surface. The flux of a transported medicament is proportionally related to the diffusivity of the environment which can be manipulated by, e.g., adjusting the pH, taking into account the ionic nature of the medicament and/or the ionic nature of the polymer or polymers included in the environment.

As used herein, the term “backing layer” or “barrier environment” or “non-adhesive polymeric environment” refers to an environment in the form of, e.g., a layer or coating or barrier layer, capable of slowing, or reducing flux of a medicament from the mucoadhesive layer into the oral cavity. In some embodiments, the backing layer may contain a second medicament intended for dissolution in the saliva. In such cases, the pH of the backing layer is adjusted, such that it impedes flux of the medicament toward the mucoadhesive layer where transmucosal absorption may occur.

As used herein, the term “unidirectional gradient” refers to a gradient which allows for the flux of a medicament (e.g., buprenorphine) through the device, e.g., through a polymeric diffusion environment, in substantially one direction, e.g., to the oral mucosa of a subject. For example, the polymeric diffusion environment may be a mucoadhesive polymeric diffusion environment in the form of a layer or film disposed adjacent to a backing layer or film. Upon oral mucosal application, a gradient is created between the mucoadhesive polymeric diffusion environment and the mucosa, and the medicament flows from the mucoadhesive polymeric diffusion environment, substantially in one direction towards the mucosa, until the backing layer dissolves.

As used herein, “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder (e.g., to alleviate pain).

The term “subject” refers to living organisms such as humans, dogs, cats, and other mammals. Administration of the medicaments included in the devices of the present invention can be carried out at dosages and for periods of time effective for treatment of a subject. In some embodiments, the subject is a human. In some embodiments, the pharmacokinetic profiles of the devices of the present invention are similar for male and female subjects.

An “effective amount” of a drug necessary to achieve a therapeutic effect may vary according to factors such as the age, sex, and weight of the subject. Dosage regimens can be adjusted to provide the optimum therapeutic response. For example, the dosage may be administered once daily, or may be divided into two individual dosages for twice daily administration. The dose may also be proportionally reduced as indicated by the exigencies of the therapeutic situation.

The term “transmucosal,” as used herein, refers to any route of administration via a mucosal membrane. Examples include, but are not limited to, buccal, sublingual, nasal, vaginal, and rectal. In some embodiments, the administration is buccal. In some embodiments, the administration is sublingual. As used herein, the term “direct transmucosal” refers to mucosal administration via the oral mucosa, e.g., buccal and/or sublingual.

As used herein, the term “water erodible” or “at least partially water erodible” refers to a substance that exhibits a water erodibility ranging from negligible to completely water erodible. The substance may readily dissolve in water or may only partially dissolve in water with difficulty over a long period of time. Furthermore, the substance may exhibit a differing erodibility in body fluids compared with water because of the more complex nature of body fluids. For example, a substance that is negligibly erodible in water may show an erodibility in body fluids that is slight to moderate. However, in other instances, the erodibility in water and body fluid may be approximately the same.

As used herein, “addiction therapy” as related to a subject includes the administration of a drug to a subject with the purpose of reducing the cravings for the addictive substance.

As used herein, the term “opioid tolerance” refers to the phenomenon in which a subject is less susceptible to the effect of an opioid drug as a consequence of its prior administration. “Acute tolerance” describes tolerance that develops very rapidly following either a single dose or a few doses of opioids given over a short period of time. “Chronic tolerance” describes the observation that opioid administration over a longer period of time produces reduced effects. Associative tolerance is best expressed with low doses of opioids at long interdose intervals and is readily modified by behavioral or environmental interventions. Nonassociative tolerance is best expressed with high doses of drugs at short interdose intervals and is not modified by behavioral or environmental interventions.

As used herein, the term “opioid tolerant subject” refers to a subject currently receiving opioid therapy. In some embodiments, the subject is taking >60 mg oral morphine/day or equianalgesic dose of another opioid for 1 week or longer, as specified in the Table 1 below.

TABLE 1

| | Approximate | |
|---|---|---|
| Opioid | Equianalgesic Oral Doses | |
| Morphine | 60 | mg |
| Tramadol | 600 | mg |
| Hydromorphone | 15 | mg |
| Oxycodone | 40 | mg |
| Hydrocodone | 60 | mg |
| Oxymorphone | 20 | mg |
| Codeine | 400 | mg |

As used herein, the term “opioid experienced subject” refers to a subject currently receiving opioid therapy. In some embodiments, the subject's daily use of opioids does not exceed the daily doses of opioids as specified in the Table 1 above.

As used herein, the term “opioid naive subject” refers to a subject who is not currently receiving opioid therapy. In some embodiments, the subject has not been exposed to opioids for 1 week or longer.

As used herein, the term “abusive” or “abusive manner” refers to uses of the devices beyond oral transmucosal administration such as by extracting the drug and injecting or snorting.

As used herein, the term “low dose of buprenorphine” refers to the daily dose less than 2.0 mg (e.g., about 200 μg to about 2000 μg, or about 240 μg to 2000 μg) of buprenorphine. As used herein, the term “high dose of buprenorphine” refers to the daily dose greater than 2.0 mg.

As used herein, the term “steady-state plasma concentration” refers to the state, wherein the fluctuation in plasma drug concentrations are the same or similar after each dose. The term “steady-state Cₘₐₓ of plasma buprenorphine concentration” refers to the state, wherein the post dose maximum plasma concentration of buprenorphine does not differ from one dose to another. The term “steady-state Cₘᵢₙ of plasma buprenorphine concentration” refers to the state, wherein the post-dose minimum plasma concentration of buprenorphine does not differ from one dose to another. In some embodiments, the devices used in the present invention provide steady-state Cₘₐₓ of plasma buprenorphine concentration in a range between about 0.1 and about 1.0 ng/mL. In another embodiment, the devices used in the present invention provide steady-state Cₘₐₓ of plasma buprenorphine concentration in a range between about 0.1 and about 0.5 ng/mL.

As used herein, the term “common opioid adverse effects” refers to adverse effects commonly experienced by the subjects taking opioid analgesics. These common opioid adverse effects include, among others, headache, constipation, nausea or vomiting, pruritus, somnolence or cognitive impairment, dry mouth, tolerance or dependence and urinary retention.

The term “mild common opioid adverse effects” refers to adverse effects that do not require a special treatment and do not interfere with the subject's daily activities. The term “moderate common opioid adverse effects” refers to adverse effects that introduce a low level of inconvenience or concern to the subjects and could interfere with daily activities, but are usually ameliorated by simple therapeutic measures. The term “severe common opioid adverse effects” refers to adverse effects that interrupt usual daily activity and typically require systemic drug therapy or other treatment.

The term “significant constipation” refers to chronic or severe constipation associated with the continuous use of morphine or other opioids.

The term “significant nausea” refers to a severe condition of nausea that is commonly known in the art. In some embodiments, the term “significant nausea” is defined with a visual analog scale (VAS) score of greater than or equal to 25 mm on a 0 to 100 mm scale.

As used herein, the term “disposed” refers to the uniform or non-uniform distribution of an element within another.

Certain aspects of the present disclosure include methods for providing pain management and/or relief to a subject in need thereof. The pain can be any pain known in the art, caused by any disease, disorder, condition and/or circumstance and can be chronic pain or acute pain. Chronic pain can arise from many sources including, but not limited to: osteoarthritic pain; chronic lower back pain; chronic pelvic pain (CPP); neuropathic pain; fibromyalgia; postoperative pain; central nervous system pain-related pain disorder; Reflex Sympathetic Dystrophy Syndrome (RSDS); repetitive stress injuries; shingles; headaches (e.g., migraine headaches); or pain related to burns, cancer (e.g., breakthrough cancer pain) or lung disease. Acute pain is typically directly related to tissue damage, and lasts for a relatively short amount of time, e.g., hours to days, or up to 7 days. In some embodiments, the chronic pain is chronic lower back pain (CLBP). In some embodiments, the chronic lower back pain is moderate to severe chronic lower back pain. In some embodiments, the pain is neuropathic pain or osteoarthritic pain. In some embodiments, the subject to be treated for moderate to severe chronic low back pain is an opioid experienced subject.

In some aspects, the present invention provides methods of managing or treating chronic pain in a subject. In some embodiments, the subject is opioid experienced, opioid tolerant or opioid naive, as defined above. In some embodiments, the subject is opioid tolerant. In some embodiments, the subject has not responded to previous treatment with the maximal doses of non-steroidal anti-inflammatory drugs (NSAIDs). The term “NSAID refractory pain” refers to pain that cannot be adequately controlled with treatment by NSAIDs at the maximum safe dosages. Once the subject is unresponsive to NSAIDs, opioid treatment is used for pain management. A variety of opioids may be used for treatment of NSAID refractory pain including buprenorphine, oxycodone, hydrocodone, oxymorphone, morphine, codeine, and fentanyl. The choice of which of the many opioids to use is dependent on a number of factors and may be adjusted according to the effect of the opioid on the patient. The phrase “first line opioid therapy” refers to the first or preferred opioid treatment used for NSAID refractory pain. Ideally, the first line opioid therapy is the opioid that will alleviate NSAID refractory pain while having only minimal side effects. In one embodiment, buprenorphine is an ideal first line opioid therapy due to having minimal side effects. For example, buprenorphine shows minimal respiratory depression, which is a significant side effect for most opioids.

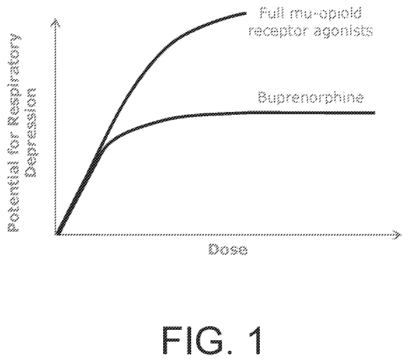

Buprenorphine is a Schedule III atypical opioid with partial μ-opioid receptor agonist activity, with high-binding affinity to the μ-opioid receptor. Without wishing to be bound to theory, the partial μ-opioid receptor agonist activity is believed to contribute to its decrease risk of respiratory depression relative to that of full μ-opioid receptor agonists (e.g., fentanyl, morphine, or oxycodone). Partial agonism refers to receptor-level activity and not analgesic efficacy, as buprenorphine has analgesic efficacy comparable to that of full μ-opioid receptor agonists. Unlike full μ-opioid receptor agonists (e.g., oxycodone), the pharmacodynamic and pharmacokinetic properties of buprenorphine contribute to a ceiling effect on respiratory depression. (See FIG. 1). This receptor activation profile of buprenorphine translates to effective analgesia and potentially greater safety than Schedule II opioids for the effective management of chronic pain.

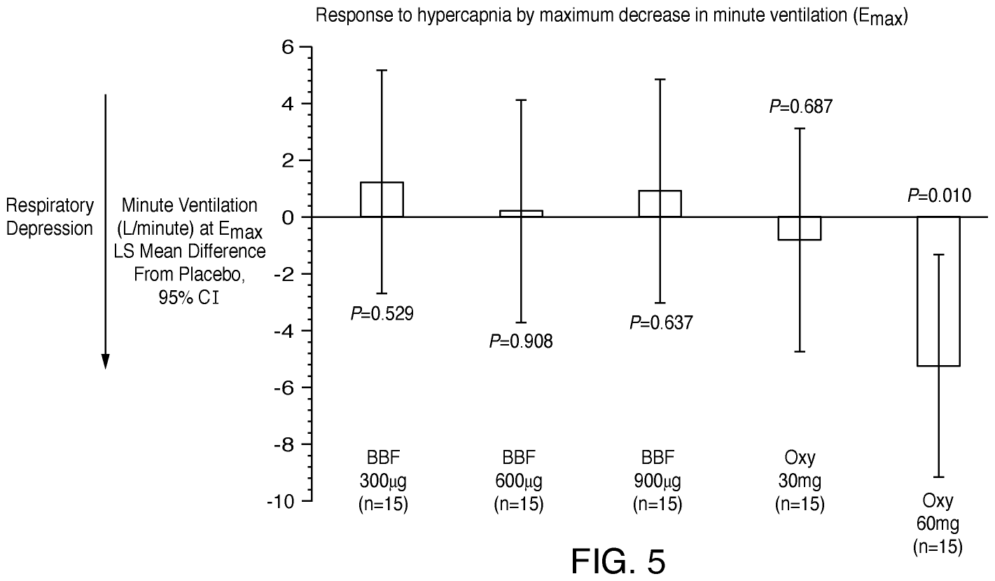

The primary cause of death related to opioid overdose is hypoxia caused by opioid-induced respiratory depression. An analysis was performed using a placebo-controlled study comparing the effects of BBF and oxycodone on respiratory drive (described herein). Respiratory depression is evaluated by measuring the ventilatory response to hypercapnia (VRH) through assessment of a decrease in maximum minute ventilation (Eₘₐₓ), relative to placebo, after administration of each dose of buprenorphine, and wherein respiratory depression is indicated when a mean difference in Eₘₐₓ, relative to placebo is clinically insignificant. FIG. 5 depicts a bar graph summary of the LS mean difference for each treatment. As can be seen in FIG. 5, relative to placebo, oxycodone decreased respiratory drive in a dose-dependent fashion (i.e., Eₘₐₓ relative to placebo was negative), whereas BBF did not impact respiratory drive at any of the doses tested.

It has also been presently found that twice daily (or once daily) administration of low doses of buprenorphine via transmucosal drug delivery devices of the present invention is associated with low incidence or absence of common opioid adverse effects associated with opioid analgesics. In some embodiments, the adverse effect is nausea. In some embodiments, the adverse effect is constipation.

Buprenorphine is considered to have a lower abuse potential than full μ-opioid receptor agonists and is therefore classified as a Schedule III drug. However, buprenorphine may induce euphoria in subjects who are not physically dependent on opioids and may be positively reinforcing. Since previous opioid studies have demonstrated a relationship between drug “liking” and pupil diameter, pupillometry was used to assess the effects of buprenorphine buccal film (BBF, BELBUCA®) and oxycodone hydrochloride (a full μ-opioid receptor agonist) on pupil diameter (described herein). For pupillometry, statistically significant miosis was slower to develop with buprenorphine buccal film than with oxycodone. The initial onset of statistically significant miosis (relative to placebo) occurred at 2 hours, 1.5 hours, and 1 hour after dosing with buprenorphine buccal film 300 μg, 600 μg, and 900 μg, respectively; and at 0.5 hours after dosing with oxycodone 30 mg or 60 mg. These results show that buprenorphine has a lower abuse potential than full μ-opioid receptor agonists.

Abuse potential may also be determined be measuring the abuse quotient (AQ) of an opioid. The “abuse quotient” is defined as Cₘₐₓ/Tₘₐₓ, where Cₘₐₓ is the maximum plasma concentration reached after administration of the opioid and Tₘₐₓ is the time that it takes to reach the maximum concentration. Opioids that are prone to abuse have a high AQ. A high AQ shows that an opioid provides a maximum dosage in a short period of time. This provides the rush desired by opioid abusers. Buprenorphine was found to have an AQ below 1 at all dosages up to 900 μg, which only had an AQ of about 0.5. This indicates that higher dosages of buprenorphine, up to at least 1800 μg, should be less prone to abuse than other opioids.

In some embodiments, transmucosal drug delivery devices of the present invention (e.g., BEMA® BioErodible MucoAdhesive Technology) are administered once daily or twice daily. In some embodiments, at least 75 μg of buprenorphine is administered transmucosally once daily. In some embodiments, from about 300 μg of buprenorphine to about 900 μg of buprenorphine is administered transmucosally once daily. In some embodiments, greater than 900 μg of buprenorphine is administered transmucosally once daily. In some embodiments, the total daily dose of buprenorphine administered is between 200 μg and 1800 μg, e.g., 200 μg, 220 μg, 240 μg, 280 μg, 300 μg, 320 μg, 350 μg, 360 μg, 400 μg, 450 μg, 480 μg, 500 μg, 550 μg, 600 μg, 620 μg, 650 μg, 700 μg, 720 μg, 750 μg, 800 μg, 860 μg, 900 μg, 960 μg, 1000 μg, 1100 μg, 1200 μg, 1250 μg, 1300 μg, 1400 μg, 1500 μg, 1600 μg, and 1800 μg.

In some embodiments, the transmucosal drug delivery devices of the present invention comprise low doses of buprenorphine. In one embodiment, the low dose of buprenorphine contained in the devices is defined as the dose of about 100 μg to about 900 μg of buprenorphine. In some embodiments, the low dose of buprenorphine comprised in the mucoadhesive device of the invention is 100 μg, 110 μg, 120 μg, 140 μg, 150 μg, 160 μg, 175 μg, 180 μg, 200 μg, 225 μg, 240 μg, 250 μg, 275 μg, 300 μg, 310 μg, 325 μg, 350 μg, 360 μg, 375 μg, 400 μg, 430 μg, 450 μg, 480 μg, 500 μg, 550 μg, 600 μg, 625 μg, 650 μg, 700 μg, 750 μg, 800 μg, 900 μg, 1000 μg, 1200 μg, 1250 μg, 1300 μg, 1400 μg, 1500 μg, 1600 μg, and 1800 μg.

### Transmucosal Pharmaceutical Delivery Device

Preparation of transmucosal pharmaceutical delivery devices have been previously described, e.g., in U.S. patent application Ser. No. 08/734,519, filed on Oct. 18, 1996, now U.S. Pat. No. 5,800,832, issued Sep. 1, 1998; U.S. patent application Ser. No. 09/144,827, filed on Sep. 1, 1998, now U.S. Pat. No. 6,159,498, issued on Dec. 12, 2000; U.S. patent application Ser. No. 11/069,089, filed on Mar. 1, 2005, Now U.S. Pat. No. 7,579,019, issued on Aug. 25, 2009; U.S. patent application Ser. No. 11/639,408, filed on Dec. 13, 2006, published as US 2007/0148097; U.S. patent application Ser. No. 11/817,915, filed on Sep. 6, 2007, published as US 2010/0015183; U.S. patent application Ser. No. 13/834,306, filed on Jul. 15, 2011, now U.S. Pat. No. 8,147,866, issued on Apr. 3, 2012; U.S. patent application Ser. No. 13/590,094, filed on Aug. 20, 2012; U.S. patent application Ser. No. 12/537,571, filed on Aug. 7, 2009, published as US 2011/0033541; and U.S. patent application Ser. No. 12/537,580, filed on Aug. 7, 2009, published as US 2011/0033542, the entire contents of which are incorporated herein by reference.

i. Mucoadhesive Layer

In some embodiments, the devices of the present invention adhere to a mucosal surface of the subject within about 5 seconds following application. In some embodiments, the devices of the present invention comprise an opioid agonist. In some embodiments, the devices of the present invention include a bioerodible- or water-erodible mucoadhesive layer, and the opioid agonist is comprised in the mucoadhesive layer. In some embodiments, the opioid agonist is buprenorphine. The dose of buprenorphine that can be incorporated into the device of the present invention depends on the desired treatment dosage to be administered and can range from about 20 μg to about 20 mg, or from about 120 μg to about 2000 μg of buprenorphine.

ii. Backing Layer

In some embodiments, the device further comprises at least one additional non-adhesive polymeric environment, e.g., a backing layer. This layer is disposed adjacent to the mucoadhesive polymeric diffusion environment, e.g., a backing layer, functions to facilitate the delivery of the opioid agonist, such as buprenorphine, to the mucosa. This additional layer may comprise the same or a different combination of polymers as the mucoadhesive polymeric diffusion environment or the non-adhesive polymeric diffusion environment.

In some embodiments, the backing layer includes an additional medicament, such as an opioid antagonist, to render the device of the invention abuse-resistant. In some embodiments, the opioid antagonist is naloxone. The dose of naloxone that can be incorporated into the backing layer of the device of the present invention can range from about 2.5 μg to about 5 mg of naloxone. In some embodiments, the amount of buprenorphine and the amount of naloxone disposed in the device are present in a ratio chosen such that the effect of buprenorphine is negated by naloxone if the mixture is injected or snorted. In some embodiments, the amount of buprenorphine and the amount of naloxone disposed in the device are present in a 4:1 w/w ratio.

### EXAMPLES

The invention will be further understood by the following examples. However, those skilled in the art will readily appreciate that the specific experimental details are only illustrative and are not meant to limit the invention as described herein, which is defined by the claims which follow thereafter.

### Example 1—Comparison of BBF to Oral Oxycodone on Respiratory Drive

A phase I placebo-controlled trial comparing the effect of buprenorphine buccal film (BBF) and oral oxycodone hydrochloride on respiratory drive.

Buprenorphine is a partial μ-opioid receptor agonist that, unlike full μ-opioid receptor agonists, has been shown to have a ceiling effect on respiratory depression. This placebo-controlled study compared the effects of buprenorphine buccal film (BBF, Belbuca®, BioDelivery Sciences International, Inc.) and oral oxycodone hydrochloride (a full μ-opioid receptor agonist) on respiratory drive.

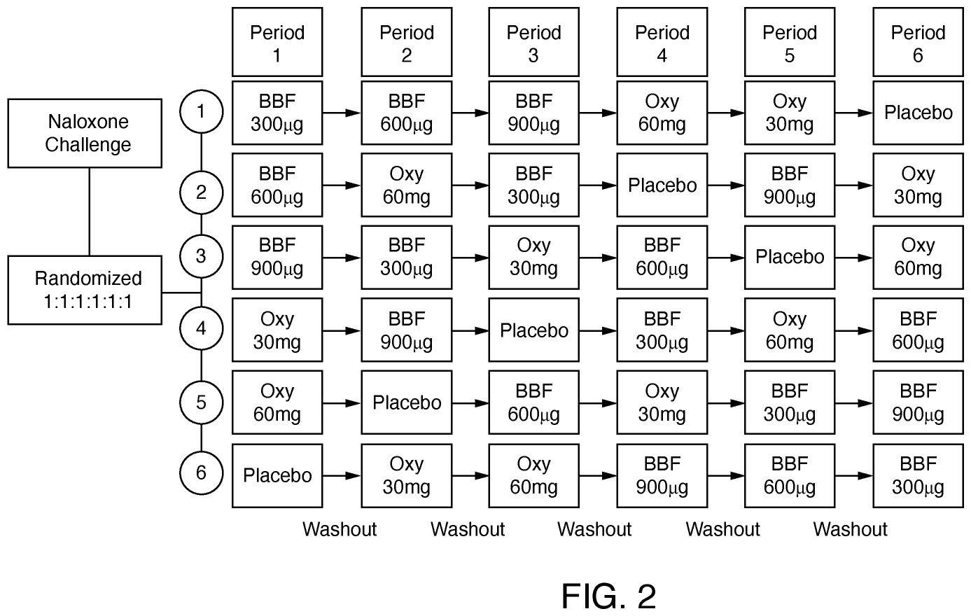

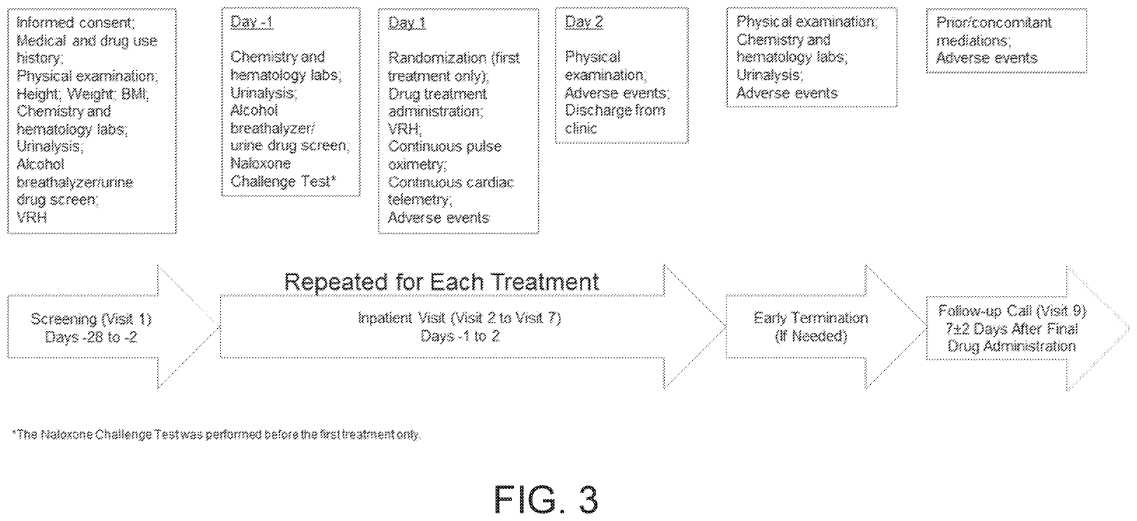

Subject Demographics and Test Design: Subjects (N=19) were males and females self-identifying as recreational drug users and determined via naloxone challenge to not be physically dependent on opioids. Effect on respiratory drive was assessed using a double-blind, double-dummy, 6-treatment, 6 period, placebo-controlled, randomized crossover design. FIG. 2 depicts a schematic diagram of the testing parameters for each group. FIG. 3 depicts a flow chart of study procedures before and during testing. A naloxone challenge test was administered to all subjects prior to randomization to confirm lack of opioid dependence. VHR tests were utilized to assess respiratory drive by evaluating breathing patterns before and immediately after administration of a hypercapnic gas mixture. A 7-day washout period was used between consecutive treatments to prevent any potential carry-over effects. Dose selection was based on an estimate of equivalent doses of buprenorphine buccal film and oxycodone required to produce a similar analgesic effect. It is estimated that 30 mg to 60 mg oxycodone falls within the analgesic efficacy range of 300 μg to 900 μg buprenorphine buccal film. Treatments were therefore selected at 300 μg buprenorphine (BBF300), 600 μg buprenorphine (BBF600), and 900 μg buprenorphine (BBF900); 30 mg oral oxycodone (Oxy30) and 60 mg oral oxycodone (Oxy60); and placebo (each separated by a 7-day washout). Table 2 is a summary of the demographics of the subjects that completed the study.

TABLE 2

| Category | Completer Population (n = 15) | |
|---|---|---|
| Sex, no. (%) | | |
| Female | 1 | (6.7%) |
| Male | 14 | (93.3%) |
| Race, no. (%) | | |
| White | 12 | (80.0%) |
| Black or African American | 1 | (6.7%) |
| Asian | 1 | (6.7%) |
| American Indian or Alaska Native | 1 | (6.7%) |
| Ethnicity, no. (%) | | |
| Hispanic or Latino | 3 | (20.0%) |
| Not Hispanic or Latino | 12 | (80.0%) |
| Age, mean (SD), y | 32.9 | (4.4) |
| BMI, mean (SD), kg/m² | 25.4 | (3.8) |

Pharmacokinetic Assessment-Respiratory Drive: Respiratory drive was evaluated by measuring the ventilatory response to hypercapnia (VHR) through assessment of the decrease in maximum minute ventilation (Eₘₐₓ) after administration of each study drug. Respiratory drive was evaluated by testing the VRH, which was performed once pre-dose and at 0.5, 1, 2, 3, and 4 hours post dose. At each time point, subjects were allowed a period of acclimation to room air to establish a regular breathing pattern. This was immediately followed by breathing of a hypercapnic gas mixture (7% CO₂, 21% 02, 72% N₂) for a 5-minute capture period, unless the subject reaches an end-tidal CO₂ of 60 mm Hg for 3 consecutive breaths, in which case the procedure was terminated. During VRH testing, minute ventilation, PEFR, and oxygen saturation were monitored continuously. For minute ventilation and PEFR, least squares (LS) mean differences between each treatment were calculated, along with differences in LS means with 95% CIs and P values. Statistical analyses were performed using a linear mixed-effects model with treatment, period, and sequence as fixed effects, and time point and treatment-by-time-point interaction as repeated fixed effects.

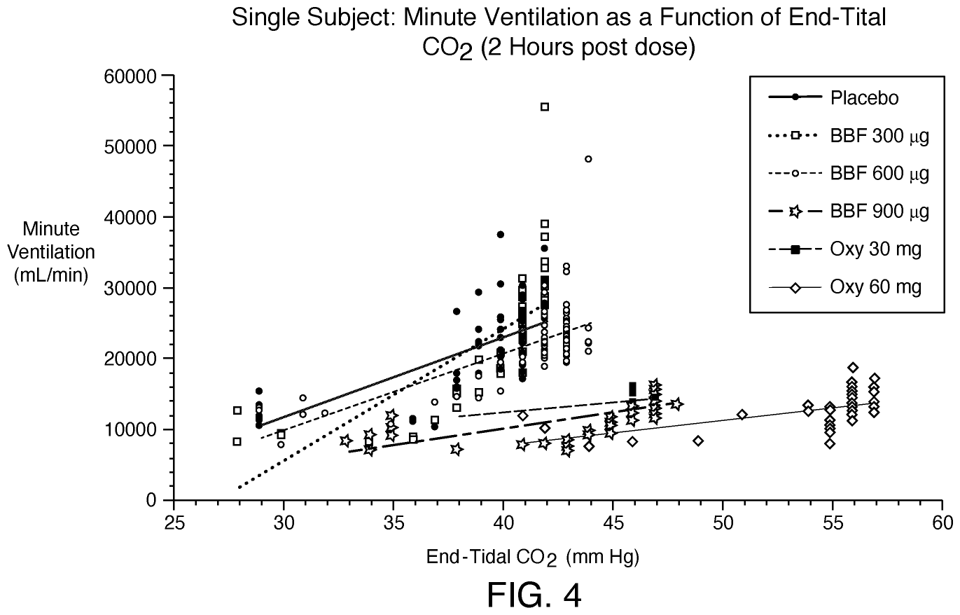

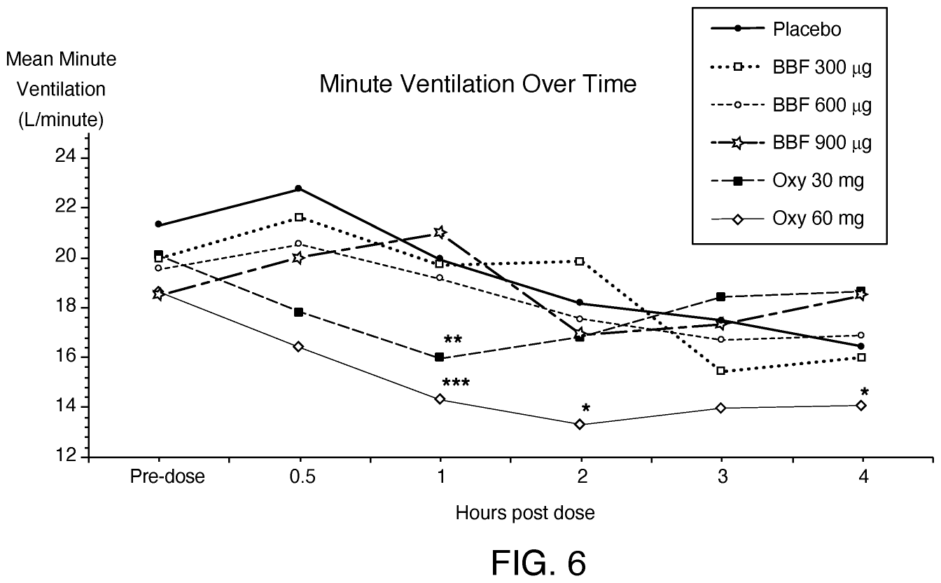

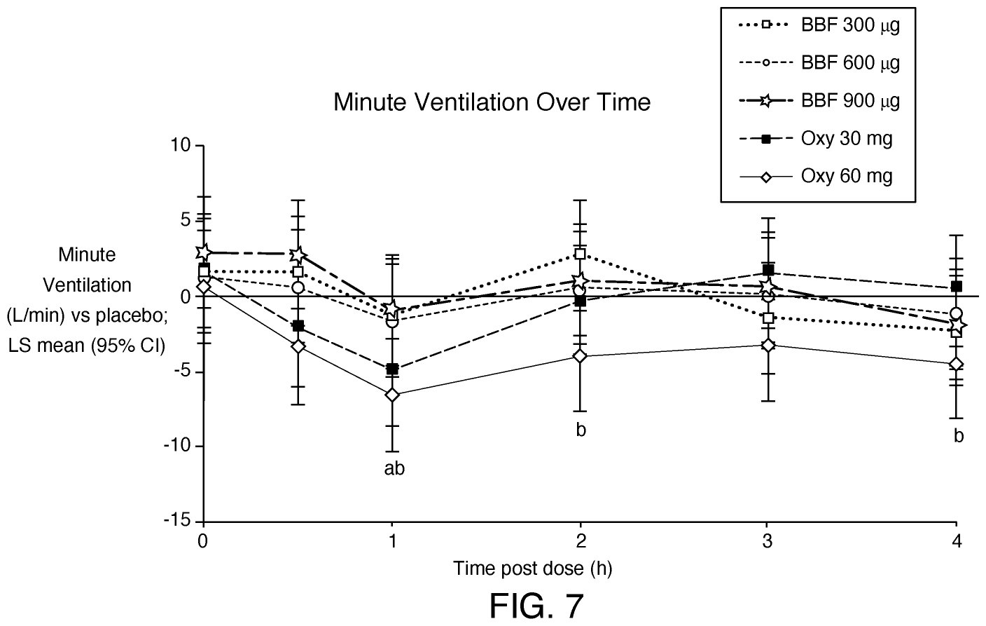

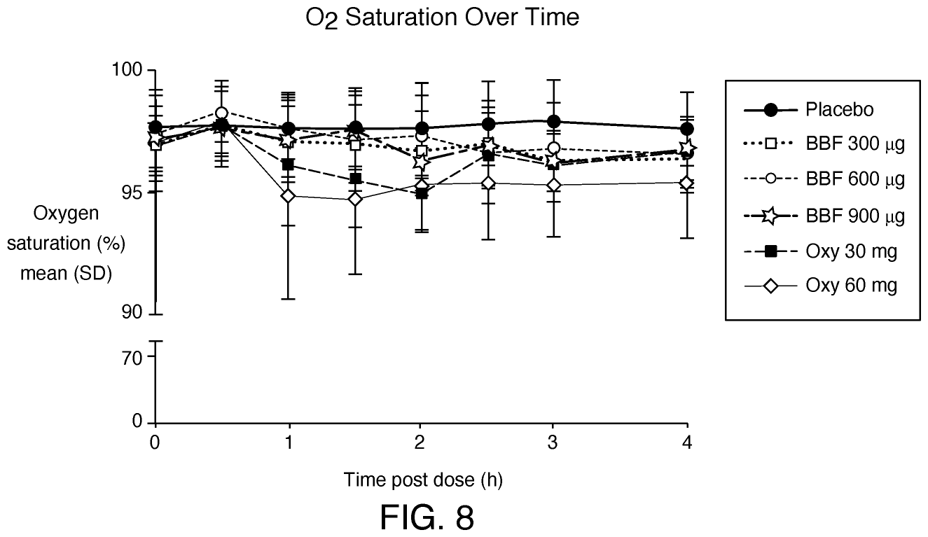

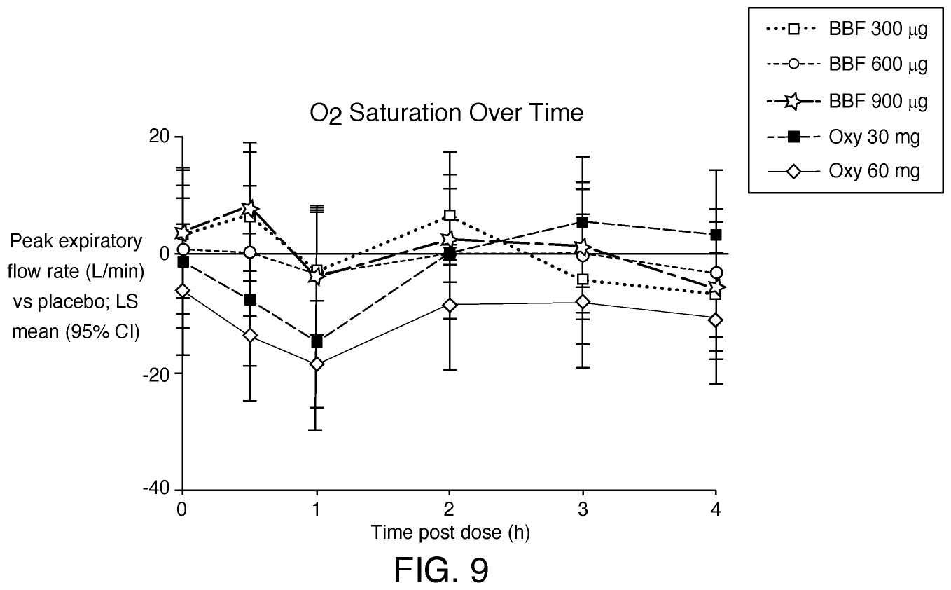

Results—Respiratory Drive: FIG. 4 depicts minute ventilation as a function of end-tidal CO₂ two hours post-dose, for a single study subject. The different effects of oxycodone and buprenorphine buccal film on minute ventilation are illustrated in FIG. 4. Minute ventilation (or respiratory minute volume or minute volume) is the volume of gas inhaled (inhaled minute volume) or exhaled (exhaled minute volume) from a person's lungs per minute. For all BBF doses, mean minute ventilation was similar to that seen with placebo across all time points. Treatment with oxycodone 30 mg led to a significant (P<0.05) decrease at one hour post-dose and oxycodone 60 mg led to a significant (P<0.05) decreases at 1 hour, 2 hour, and 4 hours post-dose, relative to placebo. FIG. 7. depicts relative minute ventilation (with respect to placebo) vs. time for all study completers. Oxycodone 30 mg and 60 mg led to a significant (P<0.05) decrease in PEFR at 1 hour post-dose, with 60 mg oxycodone also resulting in a significant (P<0.05) decrease at 30 minutes, relative to placebo. The Peak Expiratory Flow Rate (PEFR) for BBF was similar to that for placebo for all post-dose time points. FIG. 9 depicts data collected for the relative Peak Expiratory Flow Rate (PEFR), with respect to placebo, vs. time for all study completers. Mean oxygen saturation levels were mostly stable (≥95%) after treatment with each study drug. FIG. 8 depicts oxygen saturation vs. time for each study drug. One subject had an oxygen saturation level of 86% approximately 1.5 hours after receiving oxycodone 60 mg. This moderately severe adverse event was considered by the investigator to be likely related to the study drug. FIG. 6 depicts the mean minute ventilation over time for each study drug. The highest dose of BBF (900 μg) is less likely to cause respiratory depression than the highest does of oxycodone up to 4 hours after treatment. The least square mean differences in Eₘₐₓ (versus placebo) were as follows: Oxy30 (−828.5, P=0.668); Oxy60 (−5188.6, P=0.008); BBF300 (+1206.9, P=0.533); BBF600 (+245.4, P=0.896); and BBF900 (+1473.3, P=0.440). These least square mean differences are depicted in the graph in FIG. 5.

Conclusions—Respiratory Drive: Unlike oxycodone, BBF was not associated with significant decreases in minute ventilation or PEFR and did not cause any adverse reactions related to decreased oxygen saturation levels. As seen from the data, administration of oxycodone resulted in a dose-dependent decrease in respiratory drive (reduction in Eₘₐₓ). BBF did not reduce respiratory drive at any dose, including the maximum available prescription dose of 900 μg. These findings support the results from the study's primary endpoint by affirming the enhanced respiratory safety profile of BBF relative to that of the full μ-opioid receptor agonist oxycodone. Thus, BBF may be a safer treatment option than a full μ-opioid receptor agonist for patients with chronic pain.

Pharmacokinetic Assessment—Abuse Quotient: Maximum observed plasma concentration (Cₘₐₓ), time to attain maximum observed plasma concentration (Tₘₐₓ), area under the plasma concentration versus time curve from 0 to the last measurable concentration (AUC₀₋ₗₐₛₜ), and the abuse quotient (AQ, the ration of Cₘₐₓ to Tₘₐₓ) were evaluated using blood samples collected pre-dose and at 0.5, 1, 2, 3, 4, and 6 hours post-dose. Parameters were calculated using non-compartmental methods.

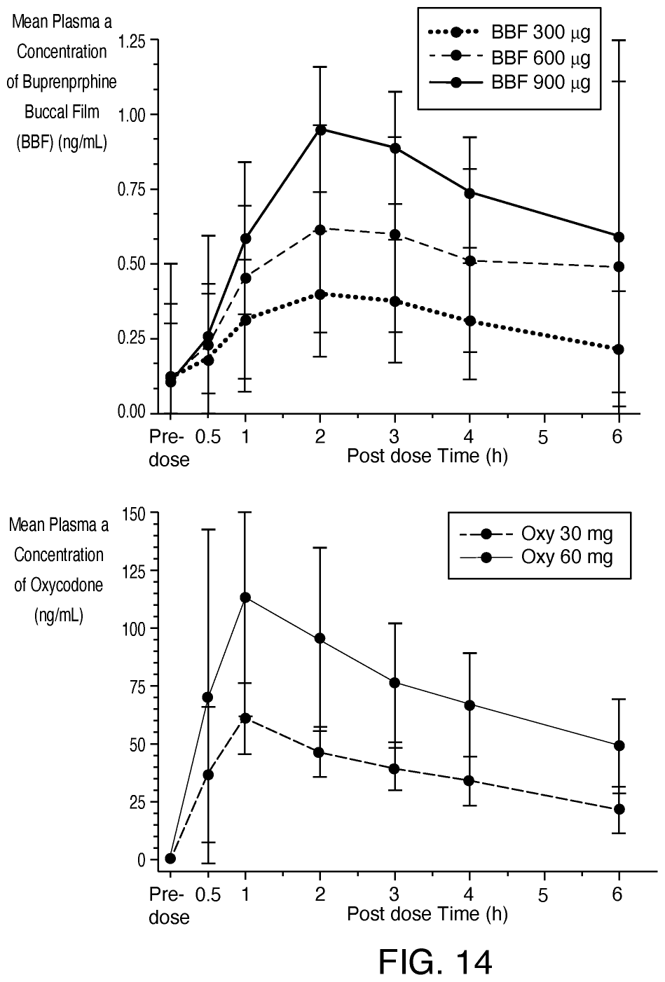

Results—Abuse Quotient: Pharmacokinetic parameters obtained from the Abuse Quotient test are presented in Table 3. FIG. 14 depicts graphs of mean plasma concentration vs. time for BBF and IR Oxycodone.

TABLE 3

| | BBF | | | IR Oxycodone | |
|---|---|---|---|---|---|
| | 300 μg | 600 μg | 900 μg | 30 mg | 60 mg |
| Parameter | (n = 15) | (n = 17) | (n = 17) | (n = 15) | (n = 16) |
| Cₘₐₓ, mean (SD) | 0.4 | 0.8 | 1.1 | 65.8 | 132 |
| ng/mL | (0.2) | (0.9) | (0.4) | (19.1) | (46.2) |
| Tₘₐₓ median | 2.2 | 3.1 | 2.2 | 1.2 | 1.2 |
| (min, max), h | (2.1, 3.2) | (1.1, 6.0) | (2.1, 6.0) | (0.6, 3.2) | (0.7, 6.0) |
| AUC₀₋ₗₐₛₜ, mean (SD), | 1.8 | 2.9 | 4.0 | 216 | 435 |
| h*ng/mL | (1.2) | (2.5) | (1.0) | (49.4) | (141) |
| AQ, mean (SD) | 0.2 | 0.3 | 0.4 | 67.4 | 110 |
| Cₘₐₓ/Tₘₐₓ | (0.1) | (0.2) | (0.1) | (39.2) | (75.3) |

The Cₘₐₓ of BBF and IR oxycodone increased proportionally with dose (Table 3, FIG. 14). IR oxycodone had a faster onset of Cₘₐₓ than BBF, as observed with Tₘₐₓ (Table 3, FIG. 14). AUC₀₋ₗₐₛₜ was numerically higher for oxycodone, but increased proportionally with dose for both study drugs (Table 3). The AQ for BBF was low (less than 1) and similar between all doses, whereas IR oxycodone had a high AQ that increased more prominently with increasing dose (Table 3, FIG. 14).

Conclusions—Abuse Quotient: BBF resulted in a slower absorption and lower AQ than IR oral oxycodone. Higher AQ is associated with greater drug liking and abuse potential. Medication selection of atypical opioids with a lower risk of drug liking and abuse potential, such as BBF, should be considered during the current opioid crisis. These data further supported the tolerability of BBF over full μ-opioid receptor agonists for the treatment of chronic pain.

Pharmacokinetic Assessment-Pupillometry: Previous opioid studies have demonstrated a relationship between drug “liking” and pupil diameter. Pupillometry was used to access the effects of buprenorphine buccal film (BBF, BELBUCA®) and oxycodone hydrochloride (a full μ-opioid receptor agonist) on pupil diameter. During VRH testing (described above) pupil diameter was determined with standard Pupillometry at the following time points: pre-dose (baseline) and 0.5 hr., 1 hr., 1.5 hrs., 2 hrs., 2.5 hrs., 3 hrs., and 4 hrs. post-dose. Statistical analyses were performed using a mixed-effects model with treatment, period, and sequence as fixed effects, and time point and treatment-by-time-point interaction as repeated fixed effects.

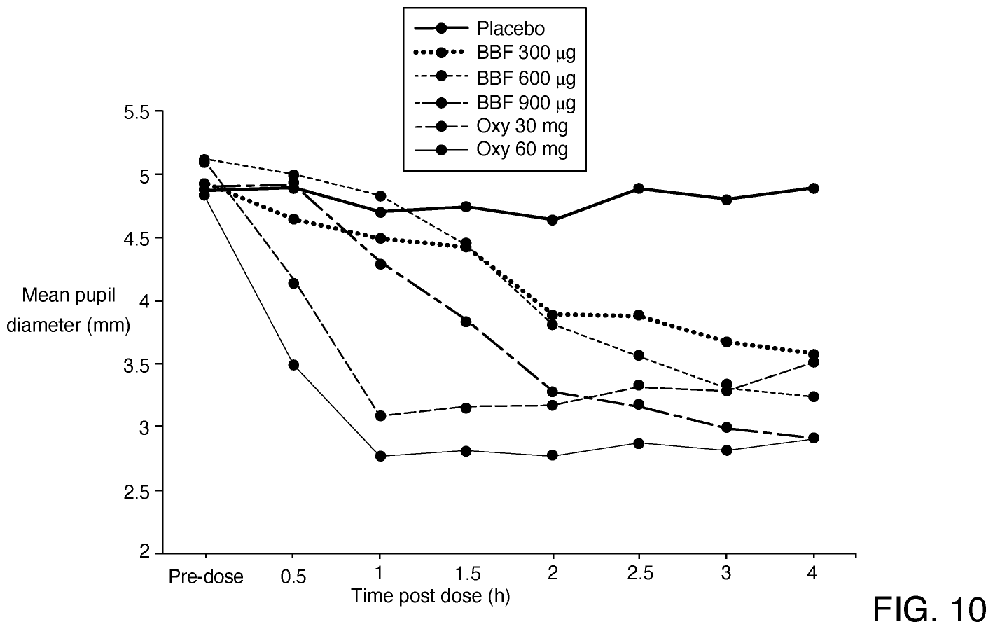

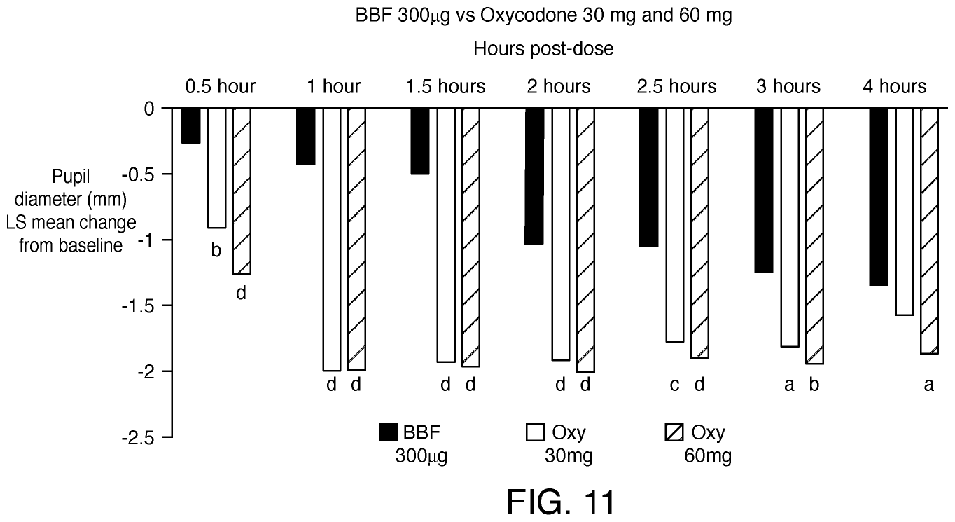

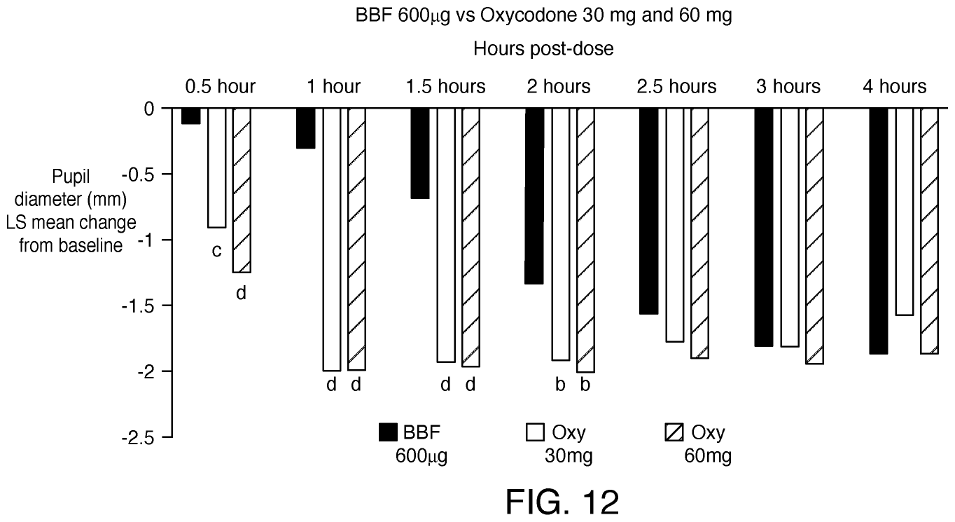

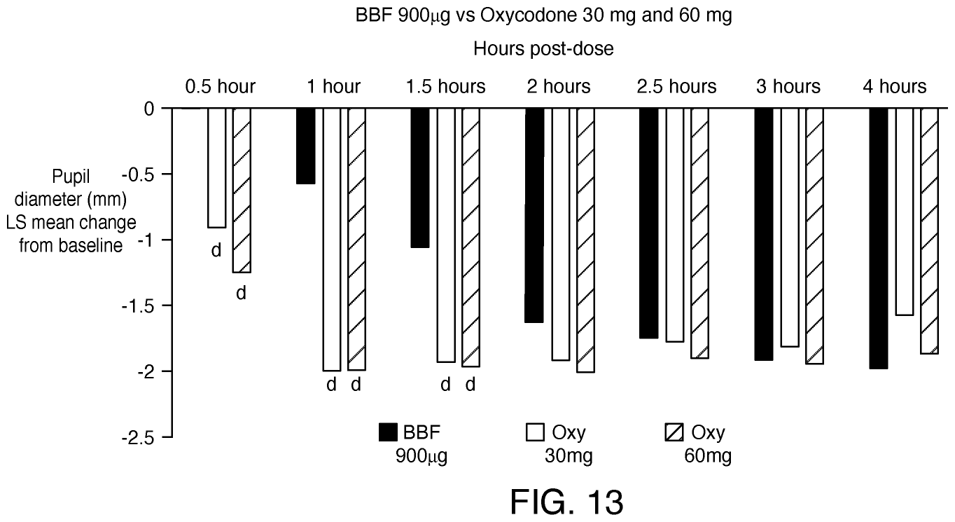

Results—Pupillometry: For pupillometry, statistically significant miosis was slower to develop with BBF than with oxycodone. The initial onset of statistically significant miosis (relative to placebo) occurred at: 2 hours after dosing for BBF 300 μg; 1.5 hours for BBF 600 μg; and 1 hour after dosing for BBF 900 μg. For oxycodone, the initial onset of statistically significant miosis (relative to placebo) occurred at 0.5 hours after dosing for both oxycodone 30 mg and oxycodone 60 mg. FIG. 10 depicts a graph of mean pupil diameter vs. hours (post-dose) for BBF 300 μg, BBF 600 μg, BBF 900 μg, oxycodone 30 mg, and oxycodone 60 mg. FIG. 11 depicts pupil diameter (mm) LS mean change from baseline over time for BBF 300 μg, oxycodone 30 mg, and oxycodone 60 mg. Miosis observed with BBF 300 μg was significantly less than seen with oxycodone 30 mg (at all time points except 4 hours post-dose) and oxycodone 60 mg (at all time points). FIG. 12 depicts pupil diameter (mm) LS mean change from baseline over time for BBF 600 μg, oxycodone 30 mg, and oxycodone 60 mg. Compared with both oxycodone doses (30 mg and 60 mg), administration of BBF 600 μg resulted in significantly less miosis for up to 2 hours post-dose. FIG. 13 depicts pupil diameter (mm) LS mean change from baseline over time for BBF 900 μg, oxycodone 30 mg, and oxycodone 60 mg. Similarly, BBF 900 μg led to significantly less miosis than either oxycodone dose did for up to 1.5 hours post-dose.

Conclusion—Pupillometry: The decrease in pupil diameter typically associated with opioid administration occurred earlier after oxycodone administration than after BBF administration. Previous studies have shown a relationship between pupil constriction and drug liking. The delayed miosis found with BBF, relative to that seen with oxycodone, may be indicative of a lower risk of drug liking and abuse potential, at least in the time period immediately following drug administration. This is also in agreement with the abuse quotient (Cₘₐₓ/Tₘₐₓ) for BBF (300 μg, 0.17) which is much lower than published estimates for oral oxycodone (30 mg: ˜15.1). Together, these results may translate to a decreased risk of drug liking and lower abuse potential for BBF compared with full u-opioid receptor agonists such as oxycodone.

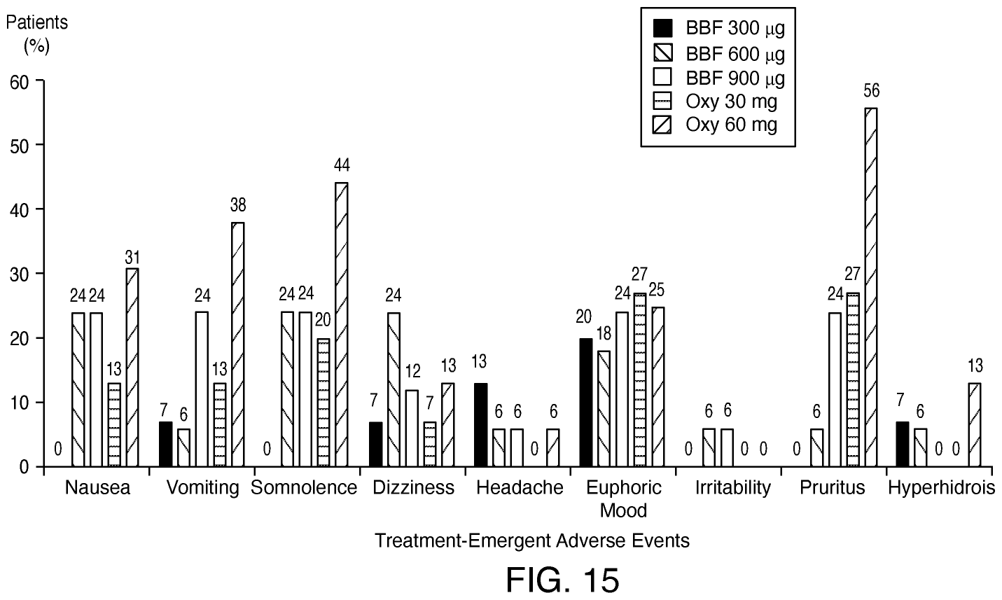

Treatment Emergent Adverse Events: FIG. 15 shows the % of patients that experienced treatment-emergent adverse events. These events included: nausea, vomiting, somnolence, dizziness, headache, euphoric mood, irritability, pruritus, and hyperhidrosis. All doses of both BBF and oxycodone produced some adverse events. Oxycodone 60 consistently produced the highest percentage of adverse events in all categories. BBF 600 μg and BBF 900 μg also consistently produced a variety of adverse events, although these events occurred, generally, in less of the population.

### Example 2—Preparation of a Buprenorphine Buccal Film (BBF)

Example 2 describes the preparation of the device of the present disclosure. Transmucosal devices are rectangular in shape with round corners. One side is a mucoadhesive layer, the other side is a backing layer. Buprenorphine is present in the mucoadhesive layer, and this side is to be placed in contact with the buccal mucosa (inside the cheek). The drug is delivered into and across the mucosa as the disc erodes in the mouth. The non-adhesive, backing layer controls the rate erosion of the disc, and minimizes the amount of buprenorphine dissolved in saliva and ultimately swallowed. Oral delivery of buprenorphine results in lower absorption due first pass metabolism. The mucoadhesive polymeric diffusion layer and the backing layer are bonded together and do not delaminate during or after application.

The mucoadhesive layer for the transmucosal devices of the present invention comprising the desired dosage of buprenorphine is prepared by mixing purified water, propylene glycol (about 4.6% total formulation, by dry weight), sodium benzoate (about 0.5% total formulation, by dry weight), methylparaben (about 0.9% total formulation, by dry weight), propylparaben (about 0.2% total formulation, by dry weight), vitamin E acetate (about 0.06% total formulation, by dry weight), citric acid (about 0.5% total formulation, by dry weight), yellow iron oxide (about 0.5% total formulation, by dry weight), monobasic sodium phosphate (about 3.4% total formulation, by dry weight). The above ingredients are added sequentially to a mixing vessel. After the components are dissolved, buprenorphine HCl (about 1.3% total formulation, by dry weight) is added, and the vessel was heated to 120° F. to 130° F. After dissolution, the polymer mixture (hydroxypropyl cellulose (about 6.8% total formulation, by dry weight), hydroxyethyl cellulose (about 20.3% total formulation, by dry weight), polycarbophil (about 6.3% total formulation, by dry weight), and carboxy methyl cellulose (about 54.3% total formulation, by dry weight)) are added to the vessel, and stirred until dispersed. Subsequently, heat is removed from the mixing vessel. As the last addition step, tribasic sodium phosphate and sodium hydroxide are added to adjust the blend to a desired pH. The blend is mixed under vacuum for a few hours. Each prepared mixture is stored in an air-sealed vessel until its use in the coating operation.

The backing layer is prepared by adding purified water to a mixing vessel followed by sequential addition of sodium benzoate (about 0.5% total formulation, by dry weight), methylparaben (about 0.4% total formulation, by dry weight), propylparaben (about 0.1% total formulation, by dry weight), citric acid (about 0.5% total formulation, by dry weight), vitamin E acetate (about 0.05% total formulation, by dry weight), sodium saccharin (about 0.5% total formulation, by dry weight). Subsequently, a mixture of the polymers hydroxypropyl cellulose (about 63% total formulation, by dry weight) and hydroxyethyl cellulose (about 32% total formulation, by dry weight) are added and stirred at a temperature between about 120° F. and 130° F., until evenly dispersed. Upon cooling to room temperature, titanium dioxide (about 2.5% total formulation, by dry weight) and peppermint oil (about 0.8% total formulation, by dry weight) are then added to the vessel and stirred. The prepared mixture is stored in an air-sealed vessel until it is ready for use in the coating operation.

The layers are cast in series onto a St. Gobain polyester liner. First, the backing layer is cast using a knife-on-a-blade coating method. The backing layer is then cured in a continuous oven at about 65° C. to 95° C. and dried. After two coating and drying iterations, an approximately 8 mil (203 to 213 micrometers) thick backing layer is obtained. Subsequently, the mucoadhesive polymeric diffusion environment is cast onto the backing layer, cured in an oven at about 65° C. to 95° C. and dried. The devices are then die-cut by kiss-cut method and removed from the casting surface.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method for the management of pain severe enough to require daily long term opioid treatment comprising:
administering at least 75 μg of buprenorphine transmucosally once daily, as a first line opioid therapy, wherein the pain is managed without substantial respiratory depression.

2. The method of claim 1, wherein administering buprenorphine comprises administering a buprenorphine buccal film.

3. The method of claim 2, wherein the buprenorphine buccal film comprises buprenorphine and naloxone.

4. The method of claim 1, wherein from about 300 μg of buprenorphine to about 900 μg of buprenorphine is administered transmucosally once daily.

5. The method of claim 1, wherein greater than 900 μg of buprenorphine is administered transmucosally once daily.

6. The method of claim 1, wherein the buprenorphine is administered once daily or twice daily.

7. The method of claim 1, wherein the respiratory depression is evaluated by measuring the ventilatory response to hypercapnia through assessment of a decrease in maximum minute ventilation (Eₘₐₓ), relative to placebo, after administration of each dose of buprenorphine, and wherein respiratory depression is indicated when a mean difference in Eₘₐₓ, relative to placebo is negative.

8. The method of claim 1, wherein the pain is caused by one or more of the following conditions: osteoarthritic pain; chronic lower back pain; chronic pelvic pain (CPP); neuropathic pain; fibromyalgia; postoperative pain; central nervous system pain-related pain disorder; Reflex Sympathetic Dystrophy Syndrome (RSDS); repetitive stress injuries; shingles; headaches; or pain related to burns, cancer or lung disease.

9. A method for treating a patient with an opioid while reducing the risk of opioid addiction, the method comprising:
administering an effective amount of buprenorphine as a first line opioid therapy to treat a disease, a symptom of a disease, or a condition of a patient; and
reducing the risk of opioid addiction by maintaining an abuse quotient of buprenorphine below 1, wherein the abuse quotient is defined as Cₘₐₓ/Tₘₐₓ.

10. The method of claim 9, wherein the buprenorphine is administered transmucosally.

11. The method of claim 10, wherein administering buprenorphine comprises administering a buprenorphine buccal film.

12. The method of claim 11, wherein the buprenorphine buccal film comprises buprenorphine and naloxone.

13. The method of claim 9, wherein administering an effective amount of buprenorphine comprises administering at least 75 μg of buprenorphine transmucosally once daily.

14. The method of claim 9, wherein administering an effective amount of buprenorphine comprises administering from about 300 μg of buprenorphine to about 900 μg of buprenorphine transmucosally once daily.

15. The method of claim 9, wherein administering an effective amount of buprenorphine comprises administering greater than 900 μg of buprenorphine transmucosally once daily.

16. The method of claim 9, wherein the buprenorphine is administered once daily or twice daily.

17. The method of claim 9, wherein the disease, the symptom of the disease, or the condition of the patient treated comprises at least one of: osteoarthritic pain; chronic lower back pain; chronic pelvic pain (CPP); neuropathic pain; fibromyalgia; postoperative pain; central nervous system pain-related pain disorder; Reflex Sympathetic Dystrophy Syndrome (RSDS); repetitive stress injuries; shingles; headaches; or pain related to burns, cancer or lung disease; depression, post-traumatic stress disorder (PTSD), or suicidal ideation.

18. A method of treating post-traumatic stress disorder (PTSD) or a symptom of PTSD comprising:
administering an effective amount of buprenorphine to a subject having PTSD, wherein PTSD or a symptom of PTSD is treated.

19. The method of claim 18, wherein administering the effective amount of buprenorphine comprises transmucosally administering the effective amount of buprenorphine.
